(19) United States
(12) Patent Application Publication
(10) Pub. No.: US 2022/0401173 A1
(43) Pub. Date: Dec. 22, 2022

(54) Notched Rim Structure for Medical Tools

(71) Applicant: Erica Alexis Whiteside, Asheville, NC (US)

(72) Inventor: Erica Alexis Whiteside, Asheville, NC (US)

(21) Appl. No.: 17/843,560

(22) Filed: Jun. 17, 2022

Related U.S. Application Data
(60) Provisional application No. 63/212,306, filed on Jun. 18, 2021.

(51) Int. Cl. A61B 50/33

(52) U.S. Cl. CPC A61B 50/33

(57) ABSTRACT

A notched rim medical organizer includes a rim structure and is configured to be integrated with or selectively seated on a receptacle and includes an internal edge and an external edge, wherein the external edge includes notches or slits spaced around the outer edge, allowing patties, sponges, gauze, cotton balls, pledgets, and other wound care and management devices to be clearly organized and secured for counting and disposal during and after medical procedures.

### RELATED APPLICATIONS

This application claims priority to and incorporates entirely by reference U.S. Provisional Patent Application Ser. No. 63/212,306 filed on Jun. 18, 2021.

### FIELD OF THE INVENTION

This invention relates to a notched rim structure for use with wound care and wound management devices in medical environments, including in surgical environments. The notched rim structure is configured to be integrated with or selectively seated on a receptacle and includes notches or slits spaced around the rim or outer edge of the structure, allowing patties, sponges, gauze, cotton balls, pledgets, and other wound care and management devices to be clearly organized and secured for counting and disposal during and after medical procedures.

### BACKGROUND OF THE INVENTION

The use of wound dressings and wound management materials has evolved as medicine and technology have advanced alongside one another. In particular, patties, gauze, cotton balls, pledgets and other wound care and management devices have been utilized for centuries in medical settings. More recently, advanced medical procedures require ever more precise medical tools and ancillary items, which may often be small in size to accommodate insertion into small incisions or small surgical fields.

Throughout medical history, errors made by medical personnel have resulted in medical items being retained within a patient cavity. Well known examples include medical personnel failing to remove tools and other medical items from a surgical cavity before closing the surgical cavity. These retained items can lead to various adverse outcomes for patients, including prolonged hospital stays, infection, unforeseen additional illnesses, and even death. From an operational perspective, such errors lead to unnecessary and unforeseen costs to hospitals and hospital staff, including patient compensation and use of medical staff in remedial actions.

As a result, modern medical practices have implemented procedures whereby medical staff must account for all tools, devices, and ancillary medical products utilized in a particular medical procedure before proper disposal of these items following conclusion of a procedure. This accounting and disposal practice prevents any medical items, tools, or devices from being retained within a patient undergoing a medical or surgical procedure. In modern medical environments, it is of paramount importance that medical personnel account for and properly dispose of these medical items at the end of a procedure, and many medical facilities currently employ specific accounting procedures as a mandatory step before a procedure may be concluded.

A number of modern medical procedures require the use of patties, gauzes, cotton balls, and other wound management devices containing a locator string attached to the body of the device. Upon removal of these devices from a patient, a medical professional will often place the devices in a basin or on a tray, where the devices will be counted to ensure all items have been accounted for before concluding the procedure and disposing of the devices. These patties, gauzes, cotton balls, and other wound management devices and their corresponding locator strings often become saturated with fluids during the course of a procedure. As a result, these devices and locator strings often adhere to and tangle with one another, causing unnecessary difficulties and delays for the medical professionals tasked with counting the devices at the conclusion of a procedure and subsequently disposing of the devices. Therefore, there exists a need for a structural device configured to secure, organize, and facilitate counting and disposal of patties, gauzes, and other wound management devices during the course of a medical procedure.

### SUMMARY OF THE INVENTION

In accordance with one form of this invention, there is provided a notched rim medical organizer. In one embodiment, the notched rim medical organizer is designed to secure and organize a plurality of medical devices having locator strings.

The notched rim medical organizer includes a notched rim structure defined by an internal edge and an external edge, wherein the external edge includes a plurality of securing notches and the notched rim structure is capable of being integrated with or selectively attached to a receptacle.

In accordance with one form of this invention, a notched rim structure's internal edge may be integrated with or selectively attached to a base structure having a base structure edge along an external perimeter of the base structure, where the rim structure's internal edge is integrally formed with the base structure edge.

In accordance with one form of this invention, there is provided a notched rim medical organizer, including a rim structure being defined by an internal edge and an external edge; the rim structure being configured in a substantially circular shape; the external edge including a plurality of securing notches; wherein the internal edge is located concentrically interior to the external edge; a lateral structure having an upper edge and a lower edge, wherein the upper edge of the lateral structure is integrated with or attached to the interior edge of the rim structure; wherein the lateral structure and the rim structure are located in different planes; a base surface being configured in a substantially circular shape; the base surface having a base surface edge along the circumference of the base surface; and the lower edge of the lateral structure being integrated with or attached to the base surface edge.

In accordance with one form of the invention, a method of securing a notched rim medical organizer device to a receptacle is provided, wherein the notched rim structure includes a base surface, an uppermost lateral surface, and a rim structure extending outwardly and substantially horizontally from the uppermost lateral surface. The rim structure includes a plurality of securing notches, wherein each securing notch is configured to organize and secure the locator string of a medical device. The method includes inserting a plurality of locator strings into securing notches, wherein one individual locator string is inserted into an individual securing notch.

### BRIEF DESCRIPTION OF THE FIGURES

For a fuller understanding of the nature of the present invention, reference should be made to the following detailed description, taken in conjunction with the accompanying figures in which:

FIG. 1 is a top view of a notched rim medical organizer including a plurality of neuro patties, in accordance with one embodiment;

FIG. 2 is an isolated view of a notched rim medical organizer in accordance with one embodiment;

FIG. 3 is a top view of a notched rim medical organizer in accordance with one embodiment;

FIG. 4 is a bottom view of a notched rim medical organizer in accordance with one embodiment;

FIG. 5 is a bottom view of a notched rim medical organizer including a plurality of neuro patties, in accordance with one embodiment;

FIG. 6 is a side perspective view of a notched rim medical organizer in accordance with one embodiment;

FIG. 7 is a bottom view of a notched rim medical organizer including a plurality of neuro patties, in accordance with one embodiment;

FIG. 8 is a side perspective view of a notched rim medical organizer including a plurality of neuro patties, in accordance with one embodiment;

FIG. 9 is a top view of a notched rim medical organizer with integrated upper rim, in accordance with one embodiment;

FIG. 10 is a top view of an attachable/detachable notched rim medical organizer, in accordance with one embodiment;

FIG. 11 is a bottom view of an attachable/detachable notched rim medical organizer, in accordance with one embodiment;

FIG. 12 is a top view of an attachable/detachable notched rim medical organizer, in accordance with one embodiment;

FIG. 13 is a bottom view of an attachable/detachable notched rim medical organizer, in accordance with one embodiment;

FIG. 14 is a side perspective view of an attachable/detachable notched rim medical organizer shown in the process of being seated on a receptacle, in accordance with one embodiment;

FIG. 15 is a top view of a notched linear strip, in accordance with one embodiment;

FIG. 16 is a bottom view of a notched linear strip, in accordance with one embodiment;

FIG. 17 is a top view of an attachable/detachable notched rim medical organizer with notched basin rim clips, in accordance with one embodiment;

FIG. 18 is a bottom view of an attachable/detachable notched rim medical organizer with notched basin rim clips, in accordance with one embodiment; and

FIG. 19 is a side perspective view of an attachable/detachable notched rim medical organizer seated on a receptacle, in accordance with one embodiment.

Like reference numerals refer to like reference parts throughout the several views of the drawings.

### DETAILED DESCRIPTION

Referring to the several views of the drawings, the notched rim medical organizer 100 is shown and described herein.

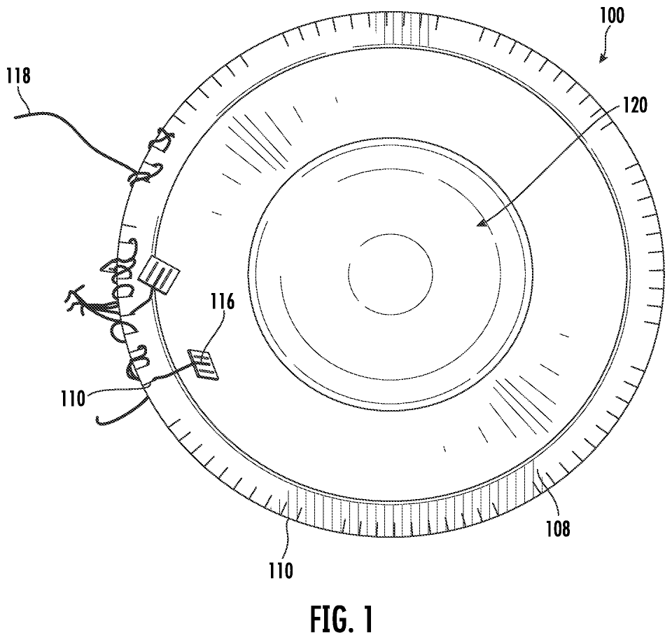

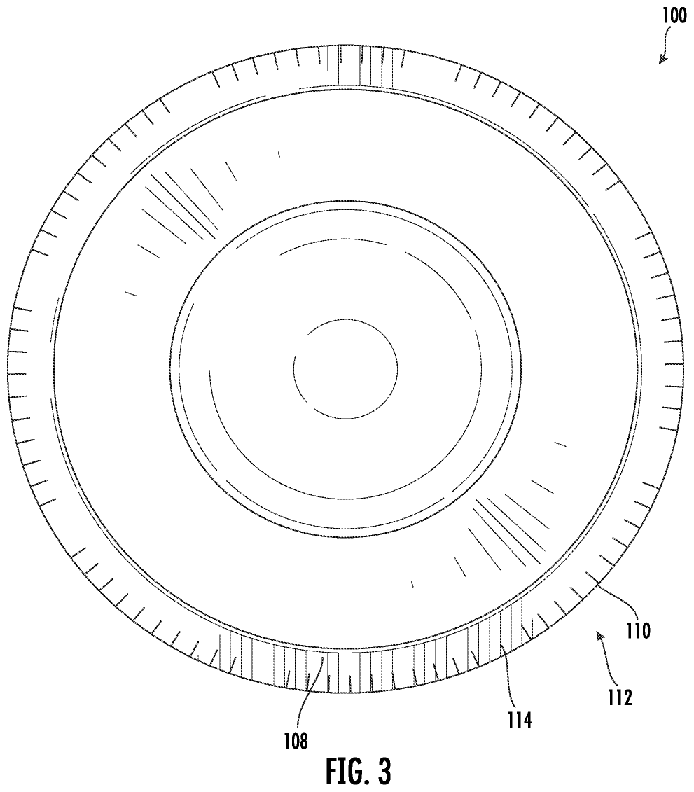

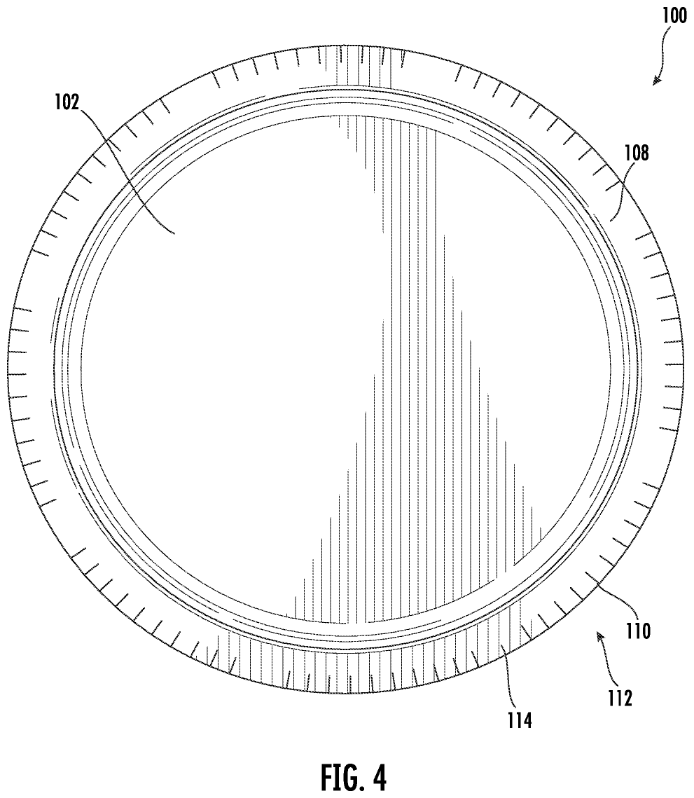

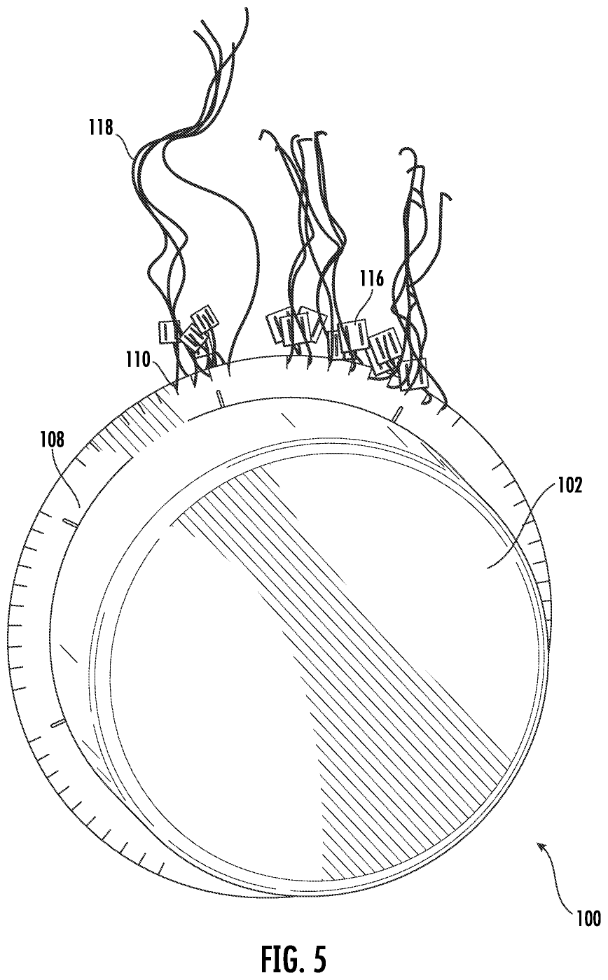

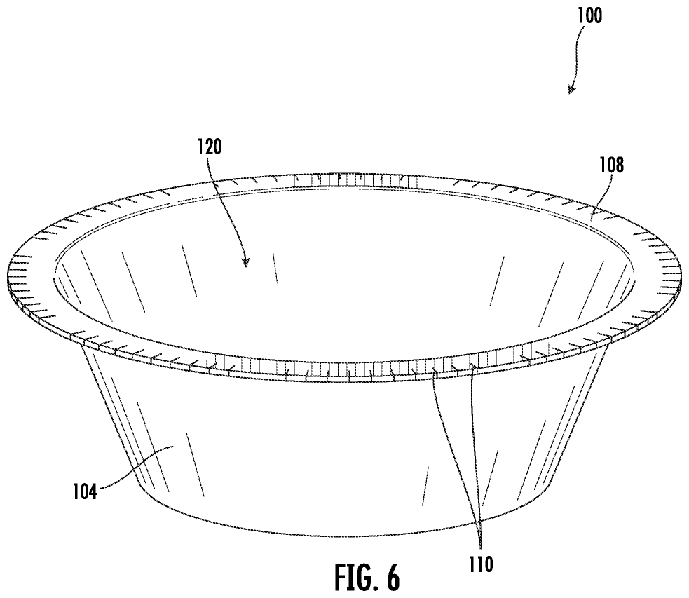

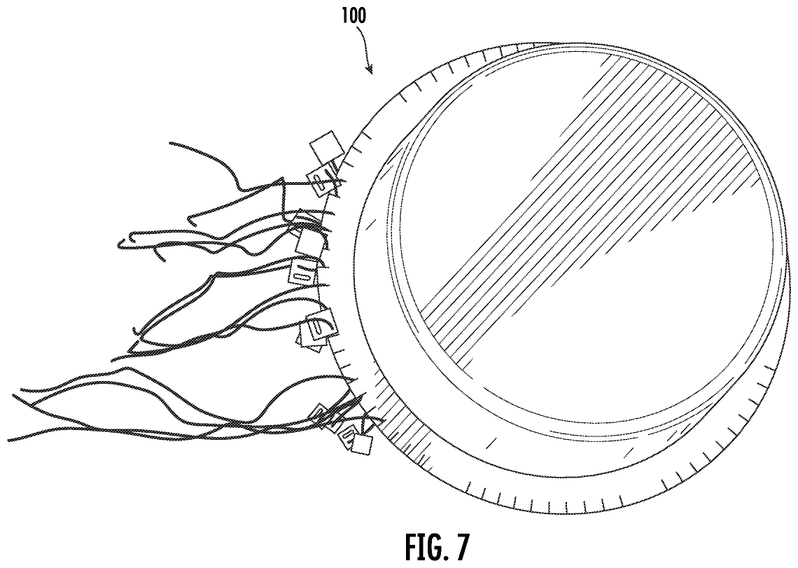

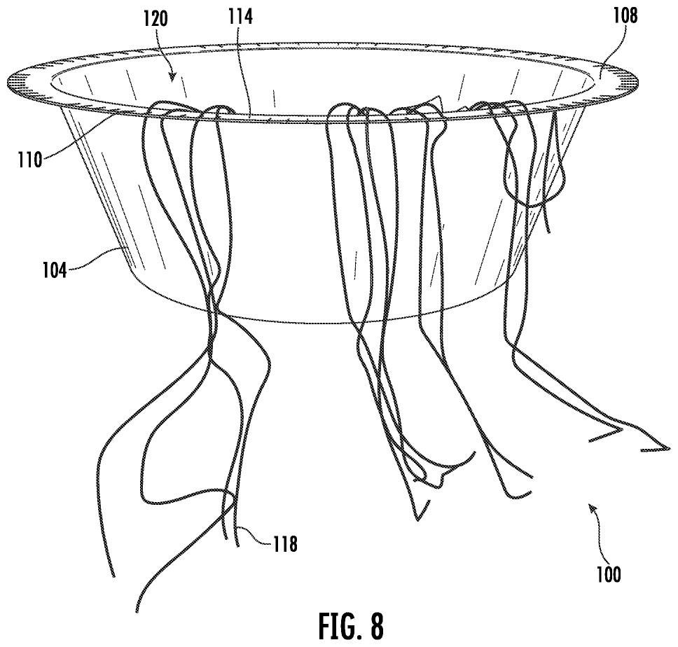

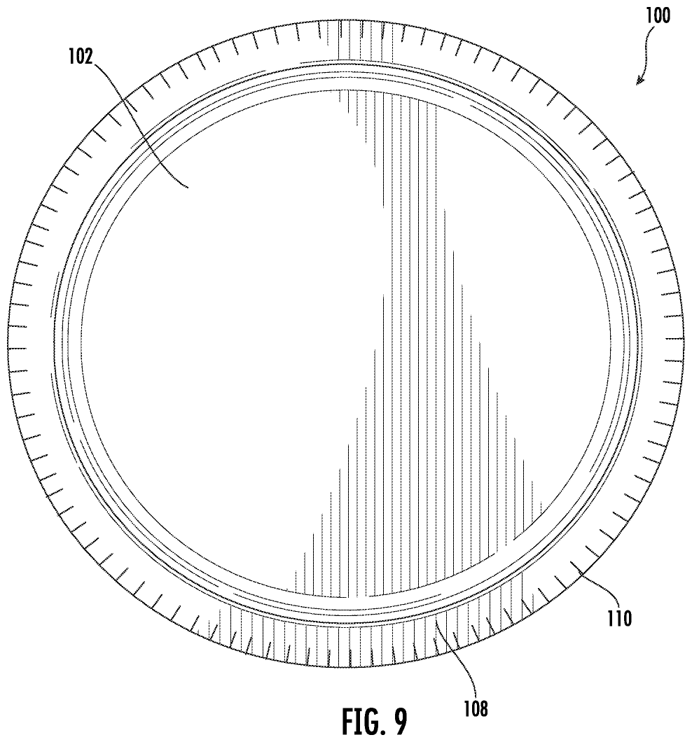

Referring initially to FIGS. 1-9, a notched rim medical organizer 100 is shown in accordance with one embodiment. The notched rim medical organizer 100 includes a base surface 102, one or more lateral surfaces 104 extending vertically, near vertically, or at other angles that are nonparallel to the base surface 102, together forming a basin volume 120. The basin volume 120 is thus bounded by the base surface 102 and one or more lateral surfaces 104. An uppermost lateral surface is located at the uppermost terminal end of the lateral surface 104. According to one embodiment, rim structure 108 extends radially and/or laterally from the uppermost lateral surface, away from the basin volume 120.

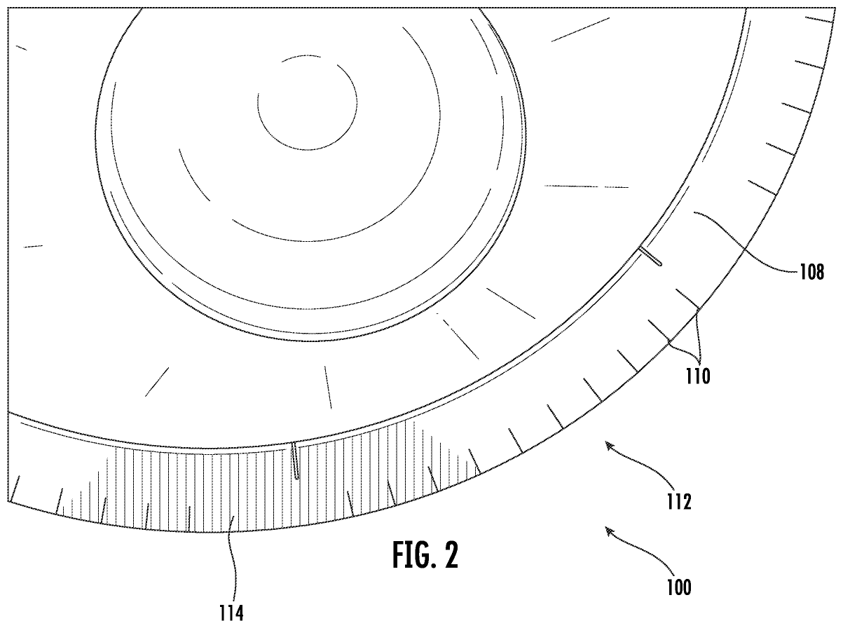

Referring now to FIG. 2, a plurality of securing notches 110 are formed or placed circumferentially along the exterior edge of the rim structure 108. The securing notches 110 may be formed in a direction parallel to the radius of a circular notched rim medical organizer 100, or in the case of other polygonal notched rim medical organizer 100 shapes, the securing notches 110 may be formed perpendicular to the outer edge of the rim structure 108 or uppermost lateral surface, wherein the edges of the securing notches 110 may be in frictional contact or near frictional contact for securing a medical device within the securing notch 110. Similarly, the securing notches 110 may be formed at alternative relative angles and may be formed in, for example, v-shaped, circular-shaped, or other alternatively-shaped notch configurations, depending on the desired configuration and use.

The securing notches 110 are configured for secured receipt of the pattie locator string 118 of a single pattie or other medical device, wherein the pattie locator string 118 is inserted into the securing notch 110 and secured in place by the frictional force between a pattie locator string 118 and a securing notch 110. By securing a single pattie or other medical device's locator string within a single securing notch 110, such medical devices are separately organized around the perimeter of the notched rim medical organizer 100. This clear visual organization allows medical personnel to safely, easily, and efficiently count the number of patties secured to a notched rim medical organizer 100 and properly dispose of the patties.

As will be appreciated by one of ordinary skill in the art, the securing notches 110 of notched rim medical organizer 100 may be used for the security and organization of numerous medical devices, including neuro patties, strung cotton balls, sponges, neuro sponges, gauze, pledgets, and other wound management devices, including those known as Telfa®, Delicot®, Uniqcot, and Cottonoid® surgical patties and sponges.

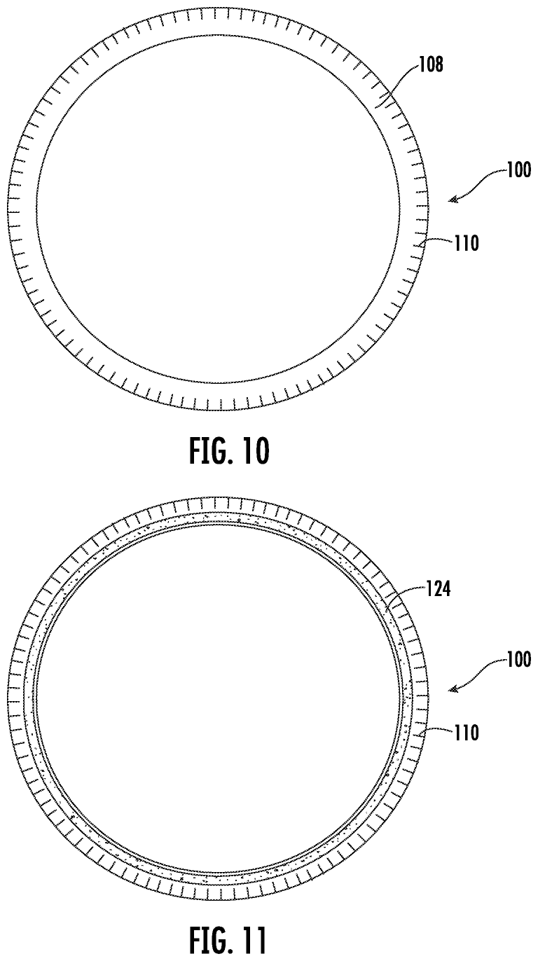

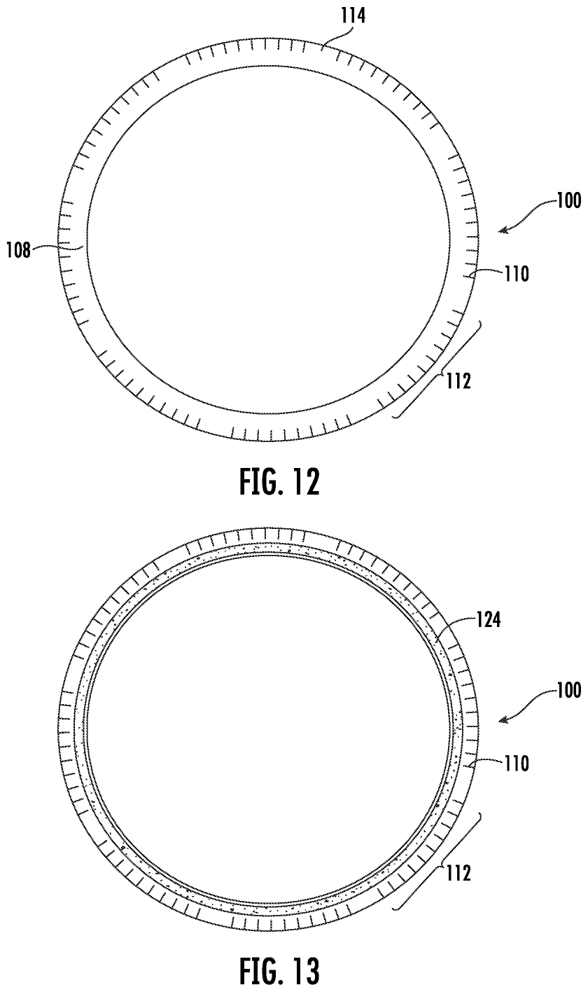

Referring now to FIGS. 10-13, according to one embodiment, the notched rim medical organizer 100 may have a rim structure 108 component that is integrated with, permanently attached to, or attachable/detachable to a standard receptacle 130, including a standard medical basin, bowl, or similar receptacle, such that the rim structure 108 may include a plurality of securing notches. As shown in FIGS. 10-13, for example, a detachable rim structure 108 may have a plurality of securing notches located along the lateral edge of the rim structure 108. The notched rim medical organizer 100 may attach to a standard receptacle 130, including a standard medical basin, bowl, or similar receptacle via an adhesive member 124 that may fully or partially cover the lower surface of the rim structure 108. According to one nonlimiting embodiment, and as illustrated in FIGS. 11 and 13, a notched rim medical organizer 100 may include, for example, an adhesive member 124 configured for attachment to, for example, a surface on a receptacle. This configuration may allow for a notched rim medical organizer 100 to be adapted for a use with a standard receptacle 130, including a standard medical basin, bowl, or similar receptacle.

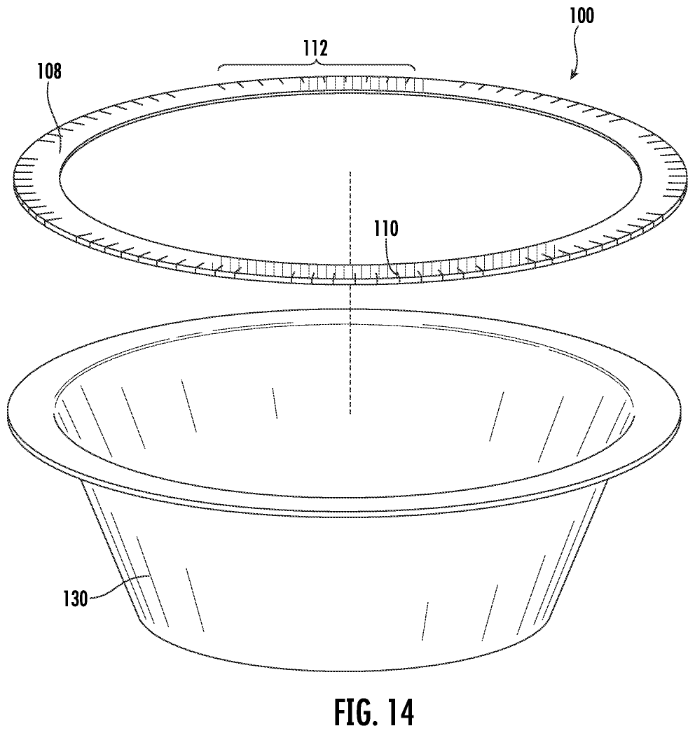

As shown in FIG. 14, and according to one nonlimiting embodiment, a notched rim medical organizer 100 may be seated atop a standard receptacle 130, including a standard medical basin, bowl, or similar receptacle. For example, an adhesive member 124 on the bottom surface of the notched rim medical organizer's 100 rim structure 108 may be used to selectively attach the notched rim medical organizer 100 to a standard receptacle 130, including a standard medical basin, bowl, or similar receptacle.

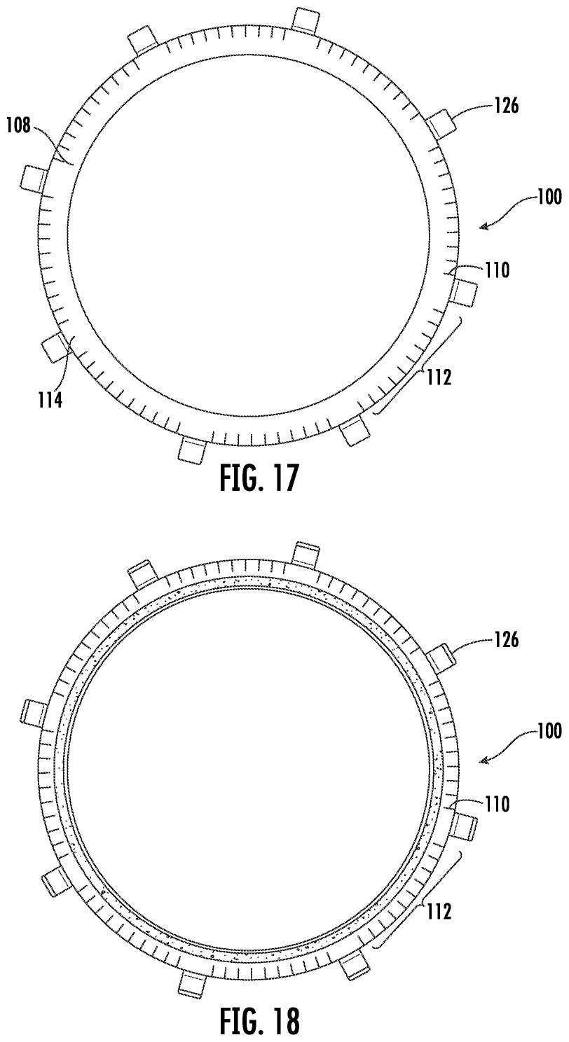

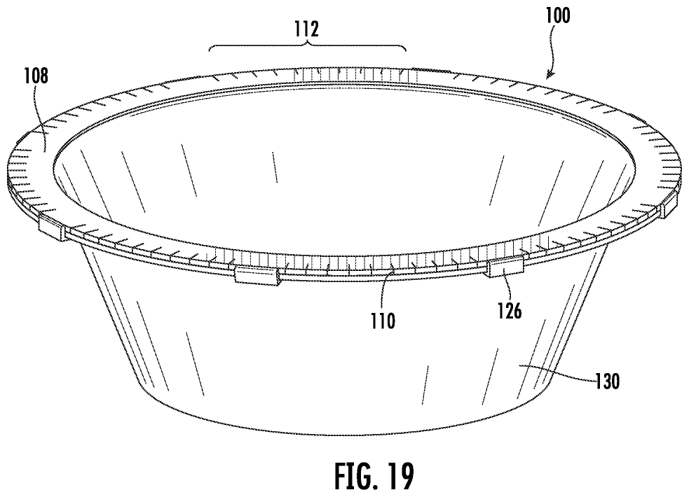

According to one nonlimiting embodiment and as illustrated in FIGS. 17-18, the notched rim medical organizer 100 may be hingedly or flexibly connected to or attached to a standard receptacle 130, including a medical basin, bowl, or similar receptacle via the use of one or more notched basin rim clips 126. It will be appreciated by one of ordinary skill that the notched basin rim clips 126 may be integrated with the notched rim medical organizer 100 with a flexible nature permitting sufficient clamping onto a standard receptacle 130, including a standard medical basin, bowl, or similar receptacle. Similarly, the notched basin rim clips 126 may be connected to the notched rim medical organizer 100 via hinge mechanism, permitting the notched basin rim clip 126 to be folded downward and clamped against a surface of a standard receptacle 130, including a standard medical basin, bowl, or similar receptacle.

According to one embodiment, and as shown, for example in FIGS. 2-4, the notched rim medical organizer 100 may include one or more rim gaps 114. In this embodiment, the rim gap 114 is adjacent to and/or in between one or more securing notch groups 112. The securing notch groups 112 include a predetermined number of adjacent securing notches 110 positioned adjacent one another and separated from other securing notch groups 112 or other securing notches 110 by one or more rim gaps 114. The rim gap 114 is thus helpful in dividing the securing notches 110 into securing notch groups 112, thereby facilitating efficient, organized counting of medical devices secured to the securing notches 110 of a notched rim medical organizer 100. For example, a notched rim medical organizer 100 may include a plurality of securing notch groups 112, wherein each securing notch group 112 includes ten securing notches 110, and wherein each securing notch group 112 is separated from adjacent securing notch groups 112 by a rim gap 114. Such an arrangement facilitates easier counting where, for example, a user may simply count the number of securing notch groups 112 that are completely filled with pattie locator strings 118 and multiply the number of filled securing notch groups 112 by the number of securing notches 110 per securing notch group 112 to quickly begin tallying the total number of pattie locator strings 118 secured in the notched rim medical organizer 100.

The securing notches 110 may be cast as part of the mold in the creation of the notched rim medical organizer 100, may be formed during a three-dimensional printing process in the creation of the notched rim medical organizer 100, or may be formed by cutting or incising along the rim structure 108 following the production or printing of the notched rim medical organizer 100.

It will be appreciated by one of ordinary skill in the art that, for example, in the embodiment of FIGS. 1-9, the notched rim medical organizer 100 may be formed without the rim structure 108 without departing from the spirit and goals of the invention. For example, securing notches 110 may be formed, printed, or cut into the uppermost lateral surface of lateral surface 104 to provide a means for securing, for example, a pattie locator string 118 or other string to the notched rim medical organizer 100.

It will be appreciated by one of ordinary skill in the art that the notched rim medical organizer 100 may be provided in numerous shapes without departing from the spirit, goals, and advantages of the invention. For example, in alternative embodiments, the notched rim medical organizer 100 may be manufactured in an oval shape, rectangular shape, kidney shape, or numerous other polygonal shapes, including shapes commonly used for receptables in medical environments. Similarly, the notched rim medical organizer 100 may be sized to be secured on or within specific stands or tables including, for example, ring stands, mayo stands, or other similar setups for use in medical procedures.

The notched rim medical organizer 100 may be composed of plastic, plastic composite, metal, metal alloy, or other suitable rigid or semi-rigid materials, including materials that may be sterilized for use in a medical environment.

According to one nonlimiting embodiment, the rim structure 108 may be of a width of approximately one- and one-half inch wide. Securing notches 110 may be spaced approximately one-half inch apart, with each rim gap 114 being approximately two inches in length. In this embodiment, each securing notch 110 is approximately one-half inch in length, extending inward, radially, from the external edge of rim structure 108.

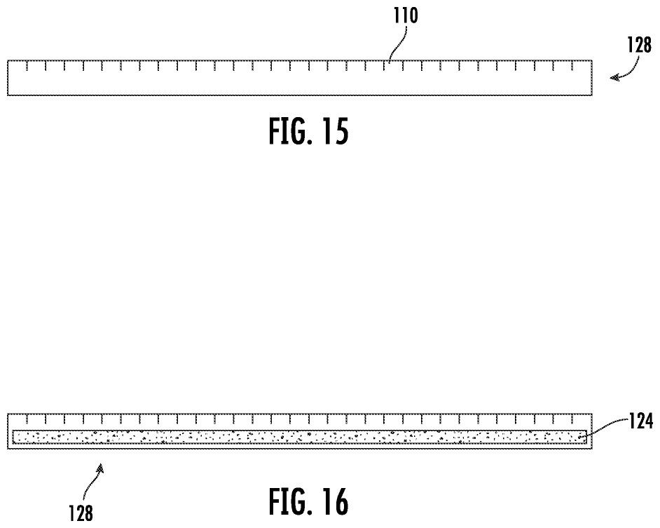

According to one nonlimiting embodiment, and as shown in FIGS. 15-16, there may be a notched linear strip 128 containing a plurality of securing notches 110 on at least one outer edge of the notched linear strip 128. The notched linear strip 128 may attach to a surface or to a standard medical basin, bowl, or similar receptable via an adhesive member that may fully or partially cover the lower surface of the notched linear strip 128.

While the present invention has been shown and described in accordance with several preferred and practical embodiments, it is recognized that departures from the instant disclosure are contemplated within the spirit and scope of the present invention.

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For brevity and/or clarity, well-known functions or constructions may not be described in detail herein.

The term “exemplary” is used herein to mean serving as an example, instance, or illustration. Any aspect or design described herein as “exemplary” is not necessarily to be construed as preferred or advantageous over other aspects or designs. Similarly, examples are provided herein solely for purposes of clarity and understanding and are not meant to limit the subject innovation or portion thereof in any manner.

The terms “for example” and “such as” mean “by way of example and not of limitation.” The subject matter described herein is provided by way of illustration for the purposes of teaching, suggesting, and describing, and not limiting or restricting. Combinations and alternatives to the illustrated embodiments are contemplated, described herein, and set forth in the claims.

For convenience of discussion herein, when there is more than one of a component, that component may be referred to herein either collectively or singularly by the singular reference numeral unless expressly stated otherwise or the context clearly indicates otherwise. For example, components N (plural) or component N (singular) may be used unless a specific component is intended. Also, the singular forms “a,” “an,” and “the” are intended to include the plural forms as well, unless expressly stated otherwise or the context indicates otherwise.

It will be further understood that the terms “includes,” “comprises,” “including,” and/or “comprising” specify the presence of stated features, steps, operations, elements, and/or components, but do not preclude the presence or addition of one or more other features, steps, operations, elements, components, and/or groups thereof unless explicitly stated otherwise or the context clearly requires otherwise. The terms “includes,” “has” or “having” or variations in form thereof are intended to be inclusive in a manner similar to the term “comprises” as that term is interpreted when employed as a transitional word in a claim.

It will be understood that when a component is referred to as being “connected” or “coupled” to another component, it can be directly connected or coupled or coupled by one or more intervening components unless expressly stated otherwise or the context clearly indicates otherwise.

The term “and/or” includes any and all combinations of one or more of the associated listed items. As used herein, phrases such as “between X and Y” and “between about X and Y” should be interpreted to include X and Y unless expressly stated otherwise or the context clearly indicates otherwise.

Terms such as “about”, “approximately”, and “substantially” are relative terms and indicate that, although two values may not be identical, their difference is such that the apparatus or method still provides the indicated or desired result, or that the operation of a device or method is not adversely affected to the point where it cannot perform its intended purpose. As an example, and not as a limitation, if a height of “approximately X inches” is recited, a lower or higher height is still “approximately X inches” if the desired function can still be performed or the desired result can still be achieved.

While the terms vertical, horizontal, upper, lower, bottom, top, and the like may be used herein, it is to be understood that these terms are used for ease in referencing the drawing and, unless otherwise indicated or required by context, does not denote a required orientation.

The different advantages and benefits disclosed and/or provided by the implementation(s) disclosed herein may be used individually or in combination with one, some or possibly even all of the other benefits. Furthermore, not every implementation, nor every component of an implementation, is necessarily required to obtain, or necessarily required to provide, one or more of the advantages and benefits of the implementation.

Conditional language, such as, among others, “can”, “could”, “might”, or “may”, unless specifically stated otherwise, or otherwise understood within the context as used, is generally intended to convey that certain embodiments preferably or optionally include certain features, elements and/or steps, while some other embodiments optionally do not include those certain features, elements and/or steps. Thus, such conditional language indicates, in general, that those features, elements and/or step may not be required for every implementation or embodiment.

The subject matter described herein is provided by way of illustration only and should not be construed as limiting the nature and scope of the subject invention. While examples of aspects of the subject invention have been provided above, it is not possible to describe every conceivable combination of components or methodologies for implementing the subject invention, and one of ordinary skill in the art may recognize that further combinations and permutations of the subject invention are possible. Furthermore, the subject invention is not necessarily limited to implementations that solve any or all disadvantages which may have been noted in any part of this disclosure. Various modifications and changes may be made to the subject invention described herein without following, or departing from the spirit and scope of, the exemplary embodiments and applications illustrated and described herein. Although the subject matter presented herein has been described in language specific to components used therein, it is to be understood that the subject invention is not necessarily limited to the specific components or characteristics thereof described herein; rather, the specific components and characteristics thereof are disclosed as example forms of implementing the subject invention. Accordingly, the disclosed subject matter is intended to embrace all alterations, modifications, and variations, that fall within the scope and spirit of any claims that are written, or may be written, for the subject invention.

What is claimed is:

1. A notched rim medical organizer comprising:
a rim structure being defined by an internal edge and an external edge;
the external edge including a plurality of securing notches;
wherein the rim structure is capable of being integrated with or selectively attached to a receptacle.

2. The notched rim medical organizer of claim 1 wherein the rim structure has a substantially planar structure.

3. The notched rim medical organizer of claim 1, wherein each of the plurality of securing notches is oriented perpendicular to the external edge.

4. The notched rim medical organizer of claim 1, wherein the rim structure is configured in a substantially circular shape.

5. The notched rim medical organizer of claim 4, wherein each of the plurality of securing notches is oriented radially to the external edge.

6. The notched rim medical organizer of claim 1, wherein each of the plurality of securing notches is grouped into one of a plurality of notch clusters, each of the plurality of notch clusters being separated by a rim gap.

7. The notched rim medical organizer of claim 6, wherein each of each of the plurality of notch clusters contains ten securing notches.

8. A notched rim medical organizer comprising:
a rim structure being defined by an internal edge and an external edge;
the external edge including a plurality of securing notches;
a base structure having a base structure edge along an external perimeter of the base structure; and
the rim structure's internal edge being integrally formed with the base structure edge.

9. The notched rim medical organizer of claim 8 wherein the rim structure has a substantially planar structure.

10. The notched rim medical organizer of claim 8, wherein each of the plurality of securing notches is oriented perpendicular to the external edge.

11. The notched rim medical organizer of claim 8, wherein the rim structure is configured in a substantially circular shape.

12. The notched rim medical organizer of claim 11, wherein each of the plurality of securing notches is oriented radially to the external edge.

13. The notched rim medical organizer of claim 8, wherein each of the plurality of securing notches is grouped into notch clusters, each of the plurality of notch clusters being separated by a rim gap.

14. The notched rim medical organizer of claim 13, wherein each of the plurality of notch clusters contains ten securing notches.

15. A notched rim medical organizer comprising:
a rim structure being defined by an internal edge and an external edge;
the rim structure being configured in a substantially circular shape;
the external edge including a plurality of securing notches;
wherein the internal edge is located concentrically interior to the external edge;
a lateral structure having an upper edge and a lower edge, wherein the upper edge of the lateral structure is integrated with or attached to the interior edge of the rim structure;
wherein the lateral structure and the rim structure are located in different planes;
a base surface being configured in a substantially circular shape;
the base surface having a base surface edge along the circumference of the base surface; and
the lower edge of the lateral structure being integrated with or attached to the base surface edge.

16. The notched rim medical organizer of claim 15, wherein the notches are oriented radially to the external edge.

17. The notched rim medical organizer of claim 15, wherein the rim structure has a substantially planar structure.

18. The notched rim medical organizer of claim 15, wherein each of the plurality of securing notches is grouped into one of a plurality of notch clusters, each of the plurality of notch clusters being separated by a rim gap.

19. The notched rim medical organizer of claim 15, wherein the base surface has a substantially planar structure.

20. The notched rim medical organizer of claim 15, wherein each of the plurality of notch clusters contains ten securing notches.
